(19) United States
(12) Patent Application Publication
(10) Pub. No.: US 2007/0213594 A1
(43) Pub. Date: Sep. 13, 2007

(54) Laryngoscope for intubation and method of use

(76) Inventor: Nghia X. Nguyen, Buena Park, CA (US)

Correspondence Address: PATENT LAW & VENTURE GROUP, 2424 S.E. BRISTOL, SUITE 300, NEWPORT BEACH, CA, 92660, US

(21) Appl. No.: 11/712,288

(22) Filed: Feb. 27, 2007

Related U.S. Application Data
(60) Provisional application No. 60/779,956, filed on Mar. 8, 2006.

(51) Int. Cl. A61B 1/267

(52) U.S. Cl. 600/190

(57) ABSTRACT

A rigid laryngoscope apparatus has a first and second elongate linear hinge portions joined in mutual coaxial rotation between proximal and distal ends. A first elongate linear handle is connected by a first handle arm to the proximal end of the first hinge portion, and a first blade is connected to the distal end of the first hinge portion. A second elongate linear handle is connected by a second handle arm to the proximal end of the second hinge portion, while a second blade is connected to the distal end of the second hinge portion. The handles are oriented in mutually parallel juxtaposition extending laterally away from the hinge portions and the blades so as to not obstruct the oral cavity.

### CROSS-REFERENCE TO RELATED APPLICATIONS

This application claims the priority date of a prior filed Provisional application having Ser. No. 60/779,956 filed on Mar. 8, 2006; the present application based on the invention described therein.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### INCORPORATION-BY-REFERENCE OF MATERIAL SUBMITTED ON A COMPACT DISC

Not applicable.

### REFERENCE TO A “MICROFICHE APPENDIX”

Not applicable.

### BACKGROUND OF THE INVENTION

1. Field of the Present Disclosure

This disclosure relates generally to laryngoscopes and more particularly to a rigid laryngoscope having laterally positioned operating handles providing improving visualization and physical access of the oral cavity.

2. Description of Related Art Including Information Disclosed Under 37 CFR 1.97 and 1.98

It is often necessary to place an endotracheal tube in a patient's airway for assisting in breathing, such as in emergency situations where a patient or victim is no longer able to breath normally, or during anesthesia or cardiac arrest to prevent aspiration of mucus, vomit, or particulate matter into the lungs. This procedure is referred to as endotracheal intubation. A laryngoscope may be used to assist in performing this procedure. Laryngoscopes may have a single blade, which may be straight or curved for guiding the tube into the trachea, or it may have two blades. A dual-bladed laryngoscope has two opposing blades that are connected by a pair of hinge points, and each blade has a handle in proximate relationship to each other for opening distal ends of the blades which then open a passageway in a patient's mouth and larynx into which an endotracheal tube may be passed. Laryngoscopes are often fitted with a light for illuminating the passageway for easier insertion of the laryngoscope and the endotracheal tube.

The intubation process is often difficult due to anatomic or pathologic differences between individuals. The most common error in intubation is placing the tube in the esophagus instead of the trachea when it becomes difficult to see the trachea or vocal cords. Failure to recognize this error can lead to fatal hypoxia in an intubated patient, and some studies have shown a failure rate of up to 30% for intubations made in the field.

In an effort to correct this problem, some laryngoscopes include fiber optics for visualizing the vocal cords for proper tube placement, however these devices are expensive and are not widely available. Other devices have been developed as alternatives to endotracheal intubation, such as the Esophogeal Obturator Airway™ (EOA), the Esophagogastric Tube Airway™ (EGTA), the Esophageal Tracheal Combitube™, and the Laryngeal Mask Airway™ (LMA). These devices are designed to allow “blind” intubation without the operator actually visualizing the vocal cords and trachea. The EOA lacks proven effectiveness and is considered dangerous by design. The EGTA is a variant of the EOA and suffers from the same drawbacks. It was a predecessor to the Combitube. Regarding the Combitube and the LMA, experts maintain that these devices should only be used temporarily until definitive airway management can be achieved through endotracheal intubation. Therefore, there is still work to be done in simplifying the endotracheal intubation process and ensuring proper and definitive airway management.

U.S. Pat. No. 5,498,231, issued to Franicevic on Mar. 12, 1996, is incorporated by reference for all purposes, including alternatives and equivalents for various elements of the presently claimed invention. The Franicevic invention is directed to a laryngoscope comprising an elongate hollow body consisting of two hollow tubes with a pair of opposed blades pivotally mounted to the distal end of the hollow body. These blades are perpendicular to the hollow body and pivotal on their axes so that the blades may assume an open or closed position. A complicated rack and pinion gear system is used to open the blades. An endotracheal tube can be passed through the hollow body and between the blades via a tube introducer. A sophisticated steering mechanism allows the tube introducer to steer the endotracheal tube into the trachea. A fiberoptic optical system with illumination is provided for observation during the intubation procedure. The primary disadvantage of this art is that the fiber optics and complicated design make it very expensive. Another disadvantage is that you must disassemble the endotracheal tube by removing the proximal fitting that connects the tube to an oxygen source in order to remove the laryngoscope, and there is no mechanism for blocking the esophagus to ensure proper tube placement in the trachea. In addition, the design of this laryngoscope makes it impossible to suction the patient without removing the entire laryngoscope first. This wastes valuable seconds when trying to establish an airway for a patient.

U.S. Pat. No. 5,938,591, issued to Minson on Aug. 17, 1999 provides a good review of the prior art on laryngoscopes and is incorporated by reference for all purposes, including alternatives and equivalents for various elements of the presently claimed invention. Minson describes a disposable, curved, dual-bladed laryngoscope with light conductive blades that open and lock apart laterally and/or radially. The blades are separated and locked apart using a ratchet mechanism in the handle that allows the device to be self-retaining in the airway. However, other than illuminating the airway, this device provides no other assistive mechanisms to increase the ease and accuracy of intubation. In addition, in order to suction a patient, a suction device must be passed through the laryngoscope first and then removed before the endotracheal tube can be inserted into the airway. If the patient vomits before the endotracheal tube is properly inserted, the tube must be removed, a suction device inserted, used, and removed, and then the endotracheal tube reintroduced through the laryngoscope. This exchanging of tubes can waste valuable seconds when seconds really count. Minson also teaches that the laryngoscope may be left in the throat after the endotracheal tube is placed in the trachea. If this occurs, his laryngoscope will have to be removed during the time one oxygen source is exchanged for another. This could add precious seconds to the time a patient is without oxygen during this exchange.

The following patents claim areas of innovations in laryngoscopes: U.S. Pat. Nos. 4,314,551; 4,384,570; 4,517,964; 5,993,383; 6,095,972; 6,174,281; 6,217,514; 6,231,505; 6,354,993; 6,471,643 and RE 37,861. However, these devices do not include features to decrease the likelihood of missed intubation, they are difficult to remove after insertion of the endotracheal tube, and they lack the ability to easily suction the patient using the same hand as the one holding the laryngoscope. In fact, none of the related art contains these features, all of which could improve the likelihood of proper tube placement and simplify the intubation process for the caregiver.

While there are suction devices that are operated independently of a laryngoscope, there are none that are integral to the laryngoscope itself. While U.S. Pat. No. 5,595,172 issued to Reese on Jan. 21, 1997, teaches suction through an endotracheal tube operated by a finger, this device is separate from the laryngoscope and in fact functions as a stylet for the endotracheal tube. This device is operated using the right hand while the left hand holds the laryngoscope. Once the endotracheal tube is in place, the suction mechanism must be removed from the tube while keeping the tube stabilized with the right hand and still holding the laryngoscope with the left hand. This process is awkward and unwieldy.

The related art described above discloses many devices for opening the airway and for visualizing as well. However, the prior art fails to disclose an airway appliance that is able to open the airway while minimizing its own obstruction of the oral cavity in general. The present disclosure distinguishes over the prior art providing advantages as described in the following summary.

### BRIEF SUMMARY OF THE INVENTION

This disclosure teaches certain benefits in construction and use which give rise to the objectives described below.

Tracheal intubation is performed in various medical conditions, for instance, in comatose or intoxicated patients who are unable to protect their airways. In such patients, the throat muscles may lose their tone so that the upper airways obstruct or collapse and air cannot easily enter into the lungs. Furthermore, protective airway reflexes such as coughing and swallowing, which serve to protect the airways against aspiration of secretions and foreign bodies, may be absent. With tracheal intubation, airway patency is restored and the lower airways can be protected from aspiration. In anesthetized patients spontaneous respiration may be decreased or absent due to the effect of anesthetics, opioids, or muscle relaxants. To enable mechanical ventilation, an endotracheal tube is often used, although there are alternative devices such as face masks or laryngeal mask airways. Intubation is common in diagnostic manipulations of the airways such as bronchoscopy and also in endoscopic operative procedures to the airways such as laser therapy or stenting of the bronchi. In intensive care medicine for patients who require respiratory support, tracheal intubation is necessary, as also in emergency medicine, particularly for cardiopulmonary resuscitation.

There are various types of tracheal tubes for oral or nasal intubation. Tubes may be either flexible or relatively stiff. Adult tubes have an inflatable cuff to seal the lower airways against air leakage and aspiration of secretions. Special double-lumen endotracheal tubes have been developed for lung and other intra-thoracic surgery. These tubes allow one-lung ventilation while the other lung can be collapsed to make surgery easier. Smaller pediatric tubes generally are uncuffed, due to concerns over blood flow to the trachea due to improper tube size or overinflation of the cuff, although some conditions require infants and children to have cuffed tubes to provide high-pressure ventilations.

Direct laryngoscopy is done with the patient lying on his or her back; the laryngoscope is inserted into the mouth to push away the tongue and lift the epiglottis so that a view of the glottis is possible. This procedure is most often employed in tracheal intubation. It is painful and extremely uncomfortable and is usually not done in conscious patients. It is performed to facilitate endotracheal intubation as part of a general anesthetic or in cardiopulmonary resuscitation following a cardiac arrest.

A rigid laryngoscope is used for direct laryngoscopy. It consists of a handle (incorporating a battery) and a blade with a source of light. There are two main types of laryngoscopes. The Macintosh blade is a curved blade and a Miller blade is straight. The Macintosh blade is easier to use while the Miller blade may give the user a better view of the vocal cords. A curved bladed instrument is defined in the Franicevic reference, while a straight bladed instrument is defined in the Zeitels reference.

The presently described laryngoscope is novel in several ways and therefore provides certain advantages. It is used together with a visualization-intubation device that permits a medical practitioner to obtain a good view of the epiglottis and its surrounding tissues. It also is capable of sealing the airway from fluid aspiration of secretions while permitting normal breathing. The laryngoscope has a pair of rigid blades, each extending from a separate handle and connected by a hinge so that they are able to be moved into mutual adjacency and, alternately, spread apart. The handles are positioned to one side of the blades so that they do not block the visual field of the medical practitioner. This arrangement works extremely well with the visualization-intubation device, also referred to as a laryngoscope, affording maximum space for movement in the oral cavity and for visualization.

A primary objective inherent in the above described apparatus and method of use is to provide advantages not taught by the prior art.

Another objective is to provide a laryngoscope that is manipulated from one side of a patient's mouth so as to leave the oral cavity relatively unobstructed;

A further objective is to provide an improved method in direct laryngoscopy.

A still further objective is to provide a rigid laryngoscope that is easily manipulated by a single person while also manipulating a visualization laryngoscope.

A still further objective is to provide a rigid laryngoscope that has proximal portions of it blades more narrow than its distal portions so as to provide effective tissue contact while still providing excellent visibility.

Other features and advantages of the present invention will become apparent from the following more detailed description, taken in conjunction with the accompanying drawings, which illustrate, by way of example, the principles of the presently described apparatus and method of its use.

### BRIEF DESCRIPTION OF THE SEVERAL VIEWS OF THE DRAWING(S)

Illustrated in the accompanying drawing(s) is at least one of the best mode embodiments of the present invention In such drawing(s):

FIG. 1 is a perspective view of the presently described airway appliance shown with manipulators spaced apart;

FIG. 2 is a further perspective view thereof showing the manipulators engaged;

FIG. 3 is a side elevation sectional view thereof showing the laryngoscope as inserted into the oral cavity with blades not spread;

FIG. 4 is a further elevation sectional view thereof showing the rigid laryngoscope as inserted and with blades spread, thereby opening the oral cavity, and further showing a flexible laryngoscope inserted into the endotracheal tube placed for visualization of a patient's airway; and

FIG. 5 is similar to FIG. 4 and showing an inflated balloon for sealing the airway thereby preventing the aspiration of fluids and further showing the withdrawal of the flexible laryngoscope from the endotracheal tube.

### DETAILED DESCRIPTION OF THE INVENTION

The above described drawing figures illustrate the described apparatus and its method of use in at least one of its preferred, best mode embodiment, which is further defined in detail in the following description. Those having ordinary skill in the art may be able to make alterations and modifications to what is described herein without departing from its spirit and scope. Therefore, it must be understood that what is illustrated is set forth only for the purposes of example and that it should not be taken as a limitation in the scope of the present apparatus and method of use.

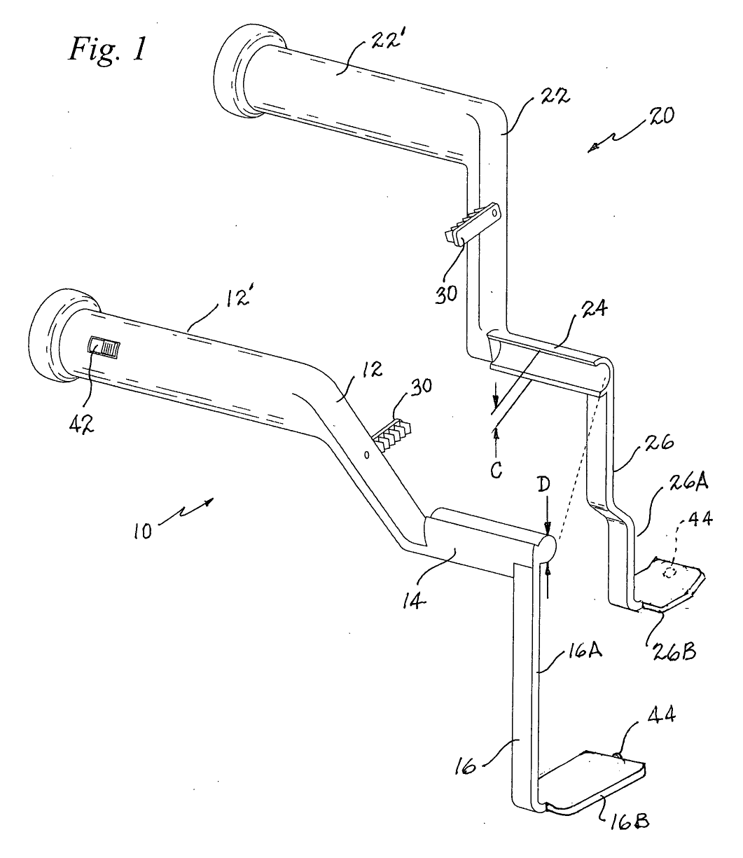

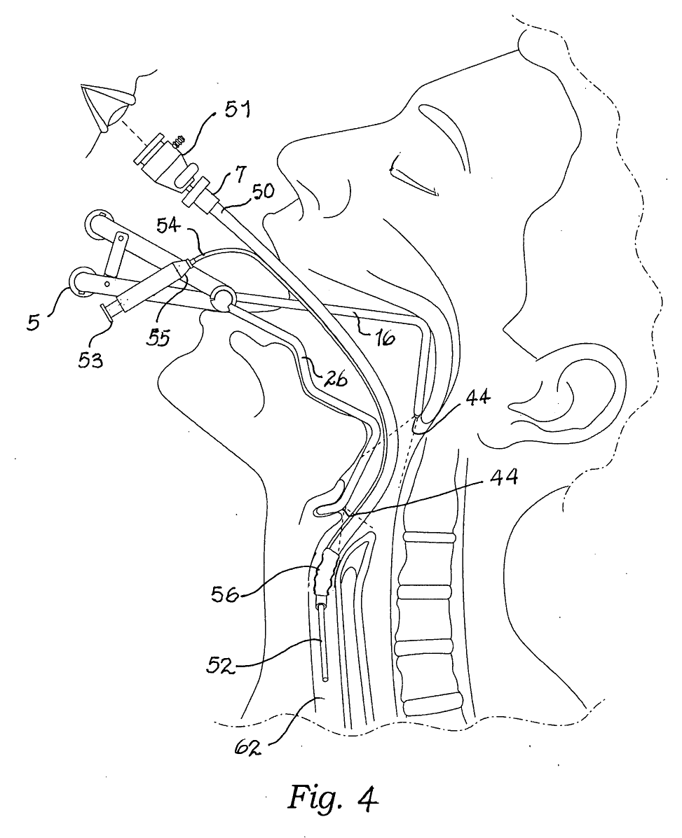

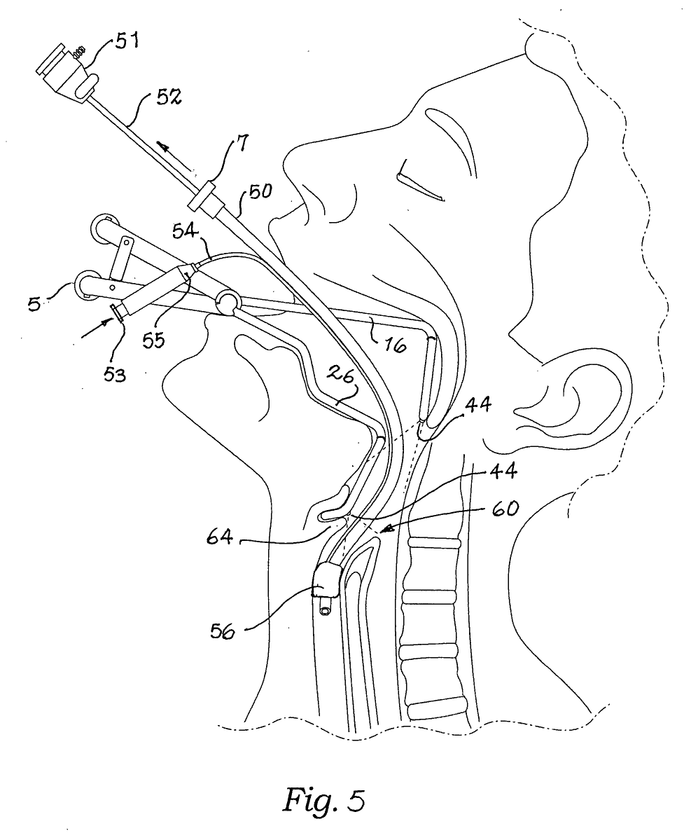

Described now in detail and referring to FIGS. 1-5, is a laryngoscope 5 of the type used for clearing, opening and illuminating the airway, with details of its construction shown as well as its method of operation and its primary application. In FIGS. 4 and 5 a flexible laryngoscope 7 of the type used for visualizing the airway and for blocking the airway from aspiration of fluids is shown. FIGS. 4 and 5 show the synergistic use of these two laryngoscopes 5 and 7, in that rigid laryngoscope 5 provides tissue clearance in the mouth, throat and airway for the more accurate and quicker placement of flexible laryngoscope 7. This can be a life-saving advantage. Rigid laryngoscope 5 is configured, as will be shown, to enable an individual, working alone, to place it properly with superior visualization during placement, and also to enable the same individual to place flexible laryngoscope 7, thereby avoiding the difficulty and confusion that may occur when such procedures require two persons as is normally the case.

Referring now to FIG. 1, laryngoscope 5 has a first integrally formed manipulator 10 formed contiguously as a first handle 12, a first hinge portion 14 and a first blade 16, the first handle 12 and the first blade 16 extending from the first hinge portion 14 in opposing directions, as shown. Laryngoscope 5 provides a second integrally formed manipulator 20 formed, as with manipulator 10, contiguously as a second handle 22, a second hinge portion 24 and a second blade 26, the second handle 22 and the second blade 26, as before, extending from the second hinge portion 24 in opposing directions.

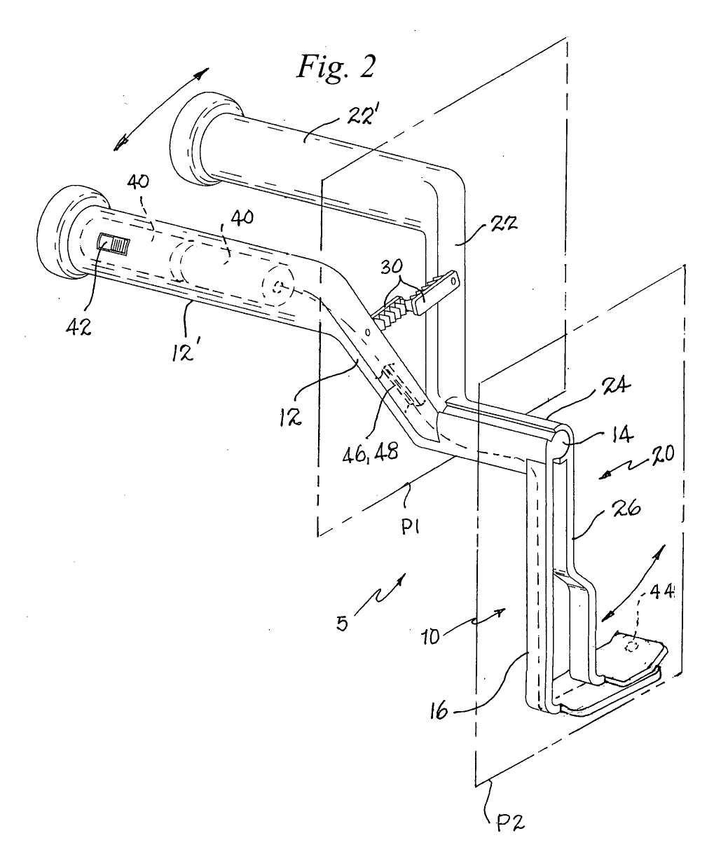

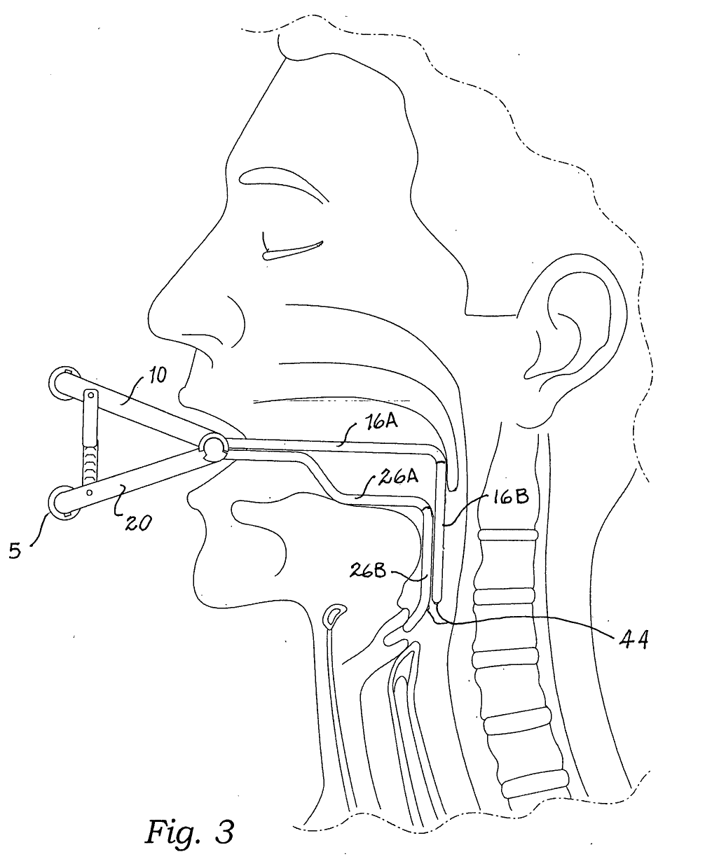

The respective handles 12 and 22, and blades 16 and 26 are spaced apart, as shown in FIG. 2, with the respective hinge portions 14 and 24 between them. Hinge portion 14 slides into hinge portion 24 and is captured therein but has enough clearance to rotate. This permits the handles 12 and 22 to move between spaced apart and adjacent positions, as shown in FIGS. 3 and 4 respectively. This movement places the blades 16 and 26 between mutually adjacent (FIG. 3) and mutually spaced apart (FIGS. 4 and 5) positions respectively. It is clearly shown in FIG. 2 that the blades 16, 26 and their respective handles 12, 22 are positioned so as to move in parallel, spaced apart planes, with the handles 12, 22 moving relative to plane P1, and the blades 16, 26 moving relative to plane P2. This enables the handles 12, 22 and their grip portions 12′, 22′ to be positioned to one side of the oral cavity so as to avoid blocking visualization during insertion, and also to allow maximum oral clearance for placement and manipulation of laryngoscope 7, as shown in FIGS. 4 and 5.

The handles 12 and 22 each provide a toothed finger 30 mounted in opposing juxtaposition, as best seen in FIG. 2, and adapted and positioned for temporarily fixing the handles 12 and 22 at selected, spaced apart, positions over an operational range of such mutual positions. FIG. 3 shows the blades 16, 26 in adjacent positions as revealed in FIG. 2, and FIG. 4 shows the blades 16, 26 in a spread apart attitude for opening the oropharynx 60 for inspection of the pharynx 62. Fingers 30 are able to secure the blades 16, 26 in the spread apart attitude, and prior to removing laryngoscope 5, fingers 30 may be disengaged to move the blades 16, 26 into mutual adjacency once more. Fingers 30 can be a set of engagable opposing teeth as shown, or any other simple mechanical engagement device as will be known to those of skill in the art and as shown in the cited references herein.

As shown in FIG. 2, either one or both of the handles 12 and 22 may engage a battery 40 and an electrical switch 42, and one or both of the blades 16, 26 may engage a miniature lamp 44. Likewise, one or both of the manipulators 10 and 20 may engage electrical conductors 46 positioned and routed within interior channels 48, so as to join the battery 40, electrical switch 42 and lamp 44 in a circuit enabled for illuminating the oropharynx. Such a circuit is well known in the art and in common use in flashlights of all kinds. Conductors 46, although depicted by a single dashed line schematically in FIG. 2, are actually a pair of thin insulated copper wires, and channels 48 are narrow passages formed within the interior of at least one of the handles 12, 22, hinge portions 14, 24 and blades 16, 26. The lamps 44 are preferably light emitting diodes (LED) and are mounted flush or slightly recessed with respect to the surface of blades 16, 26.

In the preferred embodiment, and as stated above and as best shown in FIG. 2, one of the hinge portions 14 is joined axially within the other of the hinge portions 24 wherein the hinge portions are arranged for mutual coaxial rotation. Actually, either of the hinge portions may be the inserted element, with the other being the receiving element. However, the diameter “D” of the inserted element (FIG. 1) is larger than the open space “C” of the receiving element, in this case 14 and 24, so that the manipulators 10, 20 are able to mutually rotate without easily decoupling.

Rigid laryngoscope 5, as described above may be beneficially used, as shown in FIGS. 4 and 5, for intubation when combined with flexible laryngoscope 7. Laryngoscope 7 is a commercially available medical instrument well known in the art, such as that manufactured by Mallinckrodt, Inc. of Hazelwood, Mo. It is used with a flexible plastic endotracheal tube 50. The flexible laryngoscope 7 comprises an optical fiber cable 52 which has an eyepiece 51 mounted at its proximal end. The optical fiber cable 52 is able to slide freely within the endotracheal tube 50 to position the distal end of cable 52 within tube 50 for intubation. A separate small diameter flexible tube 54 is placed within tube 50, or embedded in the wall of tube 50 or attached to the outside of the wall of tube 50 and is used to conduct a small amount of air which is typically injected using injector 53 through a valve 55 at the proximal end of the small diameter tube 54 to an annular balloon 56 mounted on the distal end of the endotracheal tube 50.

As applied, laryngoscope 5 is inserted into the throat space of a patient and adjusted as shown in FIG. 4 so as to move the epiglottis 64 frontally thereby exposing the oropharynx 60. Illumination is provided by LED lamps 44 as shown in FIG. 4 using dashed lines to define the cones of illumination. Next, laryngoscope 7 is moved into position with the distal end thereof positioned in the oropharynx 60 as shown. Air is injected into the annular balloon 56 to expand it as shown in FIG. 5, thereby sealing the oropharynx 60. The optical fiber cable 52 moves freely through the endotracheal tube 50 and since the optical cable 52 is much smaller than the inside diameter of outer tube 50, breathing is not restricted. It is now possible to remove flexible laryngoscope 7 while leaving endotracheal tube 50 inside the trachea with the annular balloon 56 inflated thereby securing the airway.

It is noted that in the above procedure the blades 16 and 26 each comprise a proximal portion 16A, 26A and a distal portion 16B and 26B respectively. In order to apply pressure in pushing against and moving tissue surfaces within the mouth and throat using blades 16 and 26, while still providing maximum visibility as well as operating space within the oral cavities, proximal portions 16A and 26A are relatively narrow compared to distal portions 16B and 26B as shown in FIG. 1.

The enablements described in detail above are considered novel over the prior art of record and are considered critical to the operation of at least one aspect of the apparatus and its method of use and to the achievement of the above described objectives. The words used in this specification to describe the instant embodiments are to be understood not only in the sense of their commonly defined meanings, but to include by special definition in this specification: structure, material or acts beyond the scope of the commonly defined meanings. Thus if an element can be understood in the context of this specification as including more than one meaning, then its use must be understood as being generic to all possible meanings supported by the specification and by the word or words describing the element.

The definitions of the words or drawing elements described herein are meant to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements described and its various embodiments or that a single element may be substituted for two or more elements in a claim.

Changes from the claimed subject matter as viewed by a person with ordinary skill in the art, now known or later devised, are expressly contemplated as being equivalents within the scope intended and its various embodiments. Therefore, obvious substitutions now or later known to one with ordinary skill in the art are defined to be within the scope of the defined elements. This disclosure is thus meant to be understood to include what is specifically illustrated and described above, what is conceptually equivalent, what can be obviously substituted, and also what incorporates the essential ideas.

The scope of this description is to be interpreted only in conjunction with the appended claims and it is made clear, here, that each named inventor believes that the claimed subject matter is what is intended to be patented.

What is claimed is:

1. A rigid laryngoscope apparatus comprising:
a first and second elongate linear hinge portions joined in mutual coaxial rotation between proximal and distal ends thereof;
a first elongate linear handle connected by a first handle arm to the proximal end of the first hinge portion, a first blade connected to the distal end of the first hinge portion, the first blade having a proximal portions and a distal portion;
a second elongate linear handle connected by a second handle arm to the proximal end of the second hinge portion, a second blade connected to the distal end of the second hinge portion;
the second blade having a proximal portions and a distal portion;
and
the handles oriented in mutually parallel juxtaposition extending away from the hinge portions and the blades, whereby with the blades inserted into a mouth, the handles are positioned to one side of the mouth.

2. The apparatus of claim 1 wherein the handles provide engagable fingers adapted for fixing the handles at selected distances apart.

3. The apparatus of claim 1 wherein at least one of the handles engages a battery and an electrical switch and at least one of the blades engages a source of illumination, the battery, electrical switch and source of illumination forming an electrical circuit for projecting illumination from at least one distal end of the laryngoscope.

4. The apparatus of claim 1 wherein the blades of the laryngoscope are generally L-shaped, and with the blades inserted into an oral cavity of a patient such that the hinge portions are adjacent to the lips of the patient, a jog in one of the blades is positioned so as to depress the tongue medially and a terminal portion of the one of the blades is configured to engage and press the epiglottis forwardly as the laryngoscope blades are separated.

5. The apparatus of claim 1 wherein the terminal portions of the blades are wider than the proximal portions of the blades.

6. A rigid laryngoscope apparatus comprising:
A linear hinge providing proximal and distal ends thereof;
a pair of handles connected by arms to the proximal end of the linear hinge, a pair of blades connected to the distal end of the linear hinge;
the blades positioned so as to move in a plane between mutually open and closed positions operated by the handles through the linear hinge; and
the handles oriented in mutually parallel juxtaposition orthogonal to the operating plane and extending away from the linear hinge and the blades to assume positions lateral thereto.

7. The apparatus of claim 6 wherein the handles provide mutually engagable fingers adapted for fixing the handles at selected distances apart.

8. The apparatus of claim 6 wherein at least one of the handles engages a battery and an electrical switch and at least one of the blades engages a source of illumination, the battery, electrical switch and source of illumination forming an electrical circuit for projecting illumination from a distal end of the laryngoscope.

9. The apparatus of claim 6 wherein the blades of the laryngoscope are generally L-shaped, and with the blades inserted into an oral cavity of a patient such that the hinge is adjacent to the lips of the patient, a jog in one of the blades is positioned so as to depress the tongue medially and a terminal portion of the one of the blades is configured to engage and press the epiglottis forwardly as the laryngoscope blades are separated.

10. The apparatus of claim 6 wherein the blades have proximal portions and terminal portions, the terminal portions of the blades being wider than the proximal portions of the blades.

11. A rigid laryngoscope apparatus comprising:
A hinge joining a pair of handles with a pair of blades;
the blades positioned so as to move in a plane between mutually open and closed positions operated by the handles through the hinge; and
the handles oriented orthogonal to the operating plane and extending away from the hinge and the blades to assume positions lateral thereto.

12. The apparatus of claim 11 wherein the handles provide mutually engagable fingers positioned for fixing the handles at selected distances apart.

13. The apparatus of claim 11 wherein at least one of the handles engages a battery and an electrical switch and at least one of the blades engages a source of illumination, the battery, electrical switch and source of illumination forming an electrical circuit for projecting illumination from a distal end of the laryngoscope.

14. The apparatus of claim 11 wherein the blades of the laryngoscope are generally L-shaped, and with the blades inserted into an oral cavity of a patient such that the hinge is adjacent to the lips of the patient, a jog in one of the blades is positioned so as to depress the tongue medially and a terminal portion of the one of the blades is configured to engage and press the epiglottis forwardly as the laryngoscope blades are separated.

15. The apparatus of claim 11 wherein the blades each have a proximal portion and a terminal portion, the terminal portions of the blades being wider than the proximal portions of the blades.
